(19) United States
(12) Patent Application Publication
(10) Pub. No.: US 2014/0155751 A1
(43) Pub. Date: Jun. 5, 2014

(54) METHOD AND SYSTEM FOR ELEMENT-BY-ELEMENT FLEXIBLE SUBARRAY BEAMFORMING

(71) Applicants: KABUSHIKI KAISHA TOSHIBA, Tokyo (JP); TOSHIBA MEDICAL SYSTEMS CORPORATION, Otawara-shi (JP)

(72) Inventors: Zoran BANJANIN, BELLEVUE, WA (US); Daniel BRUESKE, SAMMAMISH, WA (US)

(73) Assignees: KABUSHIKI KAISHA TOSHIBA, Tokyo (JP); TOSHIBA MEDICAL SYSTEMS CORPORATION, Otawara-shi (JP)

(21) Appl. No.: 13/705,864

(22) Filed: Dec. 5, 2012

(51) Int. Cl. A61B 8/00; A61B 8/08; A61B 8/14

(52) U.S. Cl. CPC A61B 8/4444; A61B 8/4488; A61B 8/145; A61B 8/5207
USPC 600/447; 600/459

(57) ABSTRACT

The subarrays are formed dynamically switching the individual output signals from the transducer elements on an element-to-element basis after the time delay circuits have individually perform on the signal signals for time delay. For example, at least one crosspoint switch flexibly connects the time-delayed outputs on the element-to-element basis in order to dynamically form predetermined sets of subarrays. The subarrays are optionally unequal in shape and or a number of transducer elements.

### FIELD

Embodiments described herein relate generally to element-by-element subarray beamforming in ultrasound diagnostic imaging systems and method of performing the same.

### BACKGROUND

As illustrated in FIG. 1, a conventional ultrasound imaging system includes a processing unit 1, a display unit 2, a cable 3 and a ultrasound transducer unit or probe 4. The probe or transducer 4 is connected to the processing unit 1 via the cable 3. The processing unit 1 generally controls the transducer unit 4 for transmitting ultrasound pulses towards a region of interest in a patient and receiving the ultrasound echoes reflected from the patient. The processing unit 1 concurrently receives from the transducer unit 4 in real time the reflected ultrasound signals for further processing so as to display on the display unit 2 an image of the region of the interest.

In detail, the transducer unit 4 further includes a predetermined number of transducer elements, which are grouped into channels for transmitting ultrasound signals and receiving the ultrasound echoes. For 2-dimensional (2D) imaging data, a number of channels generally ranges from 64 to 256. On the other hand, for 3-dimensional (3D) imaging data, a number of required channels in commercially available probes generally exceeds 1000's. In the above described conventional ultrasound imaging system, the transducer unit 4 concurrently sends the processing unit 1 via the cable 3 a large volume of reflected ultrasound data for real-time imaging while it transmits ultrasound signals and receives the ultrasound echoes.

On most of the modern Ultrasound 2D arrays beamforming is performed in two steps. The first step is called Sub Array (SA) beamforming and usually involves delaying and summing of analog signals from neighboring elements. The analog signals are generally delayed and grouped into sub arrays (SA). For example, the analog signals are summed from elements of a predetermined SA size such as 3×3, 4×3 or 4×4 adjacent elements in a 2D array that usually contains thousands of these elements. The size of SA is selected so that the number of summed signals is equal to the number of channels of the ultrasound system.

The summing in the first step is generally static. That is, the sub array size is fixed. Unfortunately, static beamforming creates several undesirable results. 2D image quality from the 2D array is inferior to that from conventional 1D and 1.5D arrays. The inferior image is due to a periodic organization of the fixed SA, which causes repeated and increased side lobes. Furtheiiuore, a particular 2D probe with beamformers of a fixed size SA limits its use for matching a particular number of channels in the ultrasound system.

On the other hand, the second step of beamforming is dynamic. Usually, digitalized signals are utilized in dynamic beamforming after the analog signals are converted. Unfortunately, although the second beamforming step may be dynamic, the extent of flexibility is limited and image quality is compromised by the statically summed analog signals from the first beamforming step. Furthermore, the beamforming step has already delayed the summed signals, and the second step requires additional complexity in dynamically beamforming the statically processed signals from the first step.

For the above reasons, it remains desirable to dynamically organize SA in order to improve image quality in 2D and 3D images using the data acquired at a 2D array. The improved image quality using dynamic SA also enhances flexibility in using a single probe with systems having a range in a number of channels.

### BRIEF DESCRIPTION OF THE DRAWINGS

FIG. 1 is a diagram illustrating one exemplary prior art ultrasound imaging system.

FIG. 2 is a schematic diagram illustrating a first embodiment of the ultrasound diagnosis apparatus according to the current invention.

FIG. 3 is a diagram illustrating a second embodiment of the probe according to the current invention.

FIG. 4 is a diagram illustrating additional components of the receiving unit and the transducer unit in the second embodiment of the probe according to the current invention.

FIG. 5 is a diagram illustrating an exemplary one-dimensional equivalent embodiment with additional components of the receiving unit and the transducer unit in the second embodiment of the probe 100-1 according to the current invention.

FIG. 6 is a diagram illustrating each of exemplary SAs to have two by two transducer elements in the third embodiment according to the current invention.

FIG. 7 is a diagram illustrating one exemplary subarray (SA) organization to improve image quality and applicability of two-dimensional (2D) arrays in one embodiment according to the current invention.

FIG. 8 is a diagram illustrating a second exemplary subarray (SA) organization to improve image quality and applicability of two-dimensional (2D) arrays in a second embodiment according to the current invention.

FIG. 9 is a diagram illustrating a third exemplary subarray (SA) organization to improve image quality and applicability of two-dimensional (2D) arrays in a third embodiment according to the current invention.

FIG. 10 is a diagram illustrating a fourth exemplary subarray (SA) organization to improve image quality and applicability of two-dimensional (2D) arrays in a fourth embodiment according to the current invention.

FIG. 11 is a diagram illustrating a fifth exemplary subarray (SA) organization to improve image quality and applicability of two-dimensional (2D) arrays in a fifth embodiment according to the current invention.

FIG. 12 is a flow chart illustrating the steps or acts involved in one embodied process with respect to the probe according to the current invention.

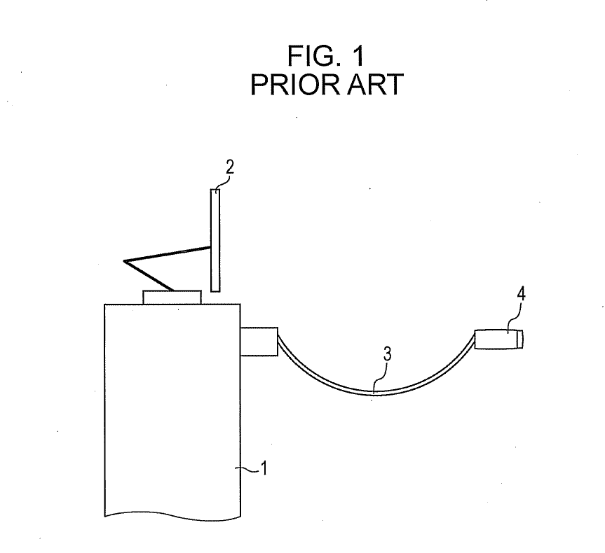

### DETAILED DESCRIPTION

Embodiments of the ultrasound imaging system according to the current invention include a probe or transducer unit, a processing unit and an optional cable connecting the probe to the processing unit. In general, the embodiments of the probe include at least some of the structures, components and elements of a conventional ultrasound probe. That is, one embodiment of the probe generates ultrasound pulses and transmits them towards a certain area of a patient. The embodiment also receives the ultrasound echoes reflected from the patient. While many embodiments of the probe are generally hand-held devices, some are not hand-held devices.

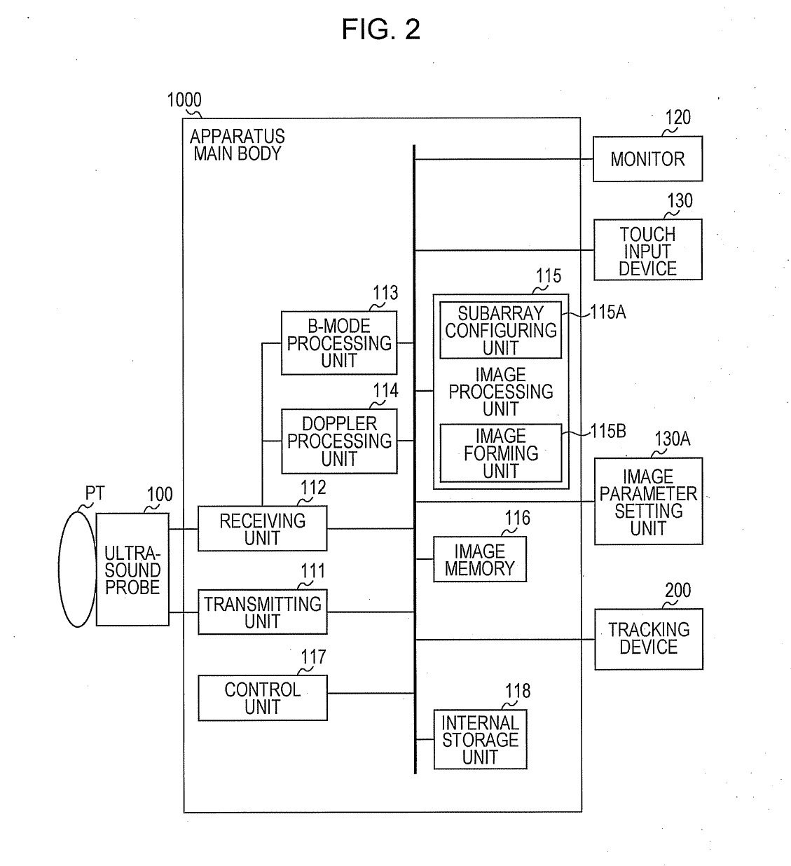

According to the current invention, exemplary embodiments of the ultrasound diagnosis apparatus will be explained below in detail with reference to the accompanying drawings. Now referring to FIG. 2, a schematic diagram illustrates a first embodiment of the ultrasound diagnosis apparatus according to the current invention. The first embodiment includes an ultrasound probe 100, a monitor 120, a touch input device 130 and an apparatus main body 1000. One embodiment of the ultrasound probe 100 further includes a plurality of transducer elements such as piezoelectric vibrators, which generate ultrasound based on a driving signal supplied from a transmitting unit 111 housed in the apparatus main body 1000.

As ultrasound is transmitted from the transducer elements such as piezoelectric vibrators in the ultrasound probe 100 to the subject Pt, the transmitted ultrasound is consecutively reflected by discontinuity planes of acoustic impedance in internal body tissue of the subject Pt and is also received as a reflected wave signal by the piezoelectric vibrators of the ultrasound probe 100. The amplitude of the received reflected wave signal depends on a difference in the acoustic impedance of the discontinuity planes that reflect the ultrasound. For example, when a transmitted ultrasound pulse is reflected by a moving blood flow or a surface of a heart wall, a reflected wave signal is affected by a frequency deviation. That is, due to the Doppler effect, the reflected wave signal is dependent on a velocity component in the ultrasound transmitting direction of a moving object.

The apparatus main body 1000 ultimately generates signals representing an ultrasound image. The apparatus main body 1000 controls the transmission of ultrasound from the probe 100 towards a region of interest in a patient as well as the reception of a reflected wave at the ultrasound probe 100. The apparatus main body 1000 includes a transmitting unit 111, a receiving unit 112, a B-mode processing unit 113, a Doppler processing unit 114, an image processing unit 115, an image memory 116, a control unit 117 and an internal storage unit 118, all of which are connected via internal bus.

The transmitting unit 111 includes a trigger generating circuit, a delay circuit, a pulsar circuit and the like and supplies a driving signal to the ultrasound probe 100. The pulsar circuit repeatedly generates a rate pulse for forming transmission ultrasound at a certain rate frequency. The delay circuit controls a delay time in a rate pulse from the pulsar circuit for utilizing each of the piezoelectric vibrators so as to converge ultrasound from the ultrasound probe 100 into a beam and to determine transmission directivity. The trigger generating circuit applies a driving signal (driving pulse) to the ultrasound probe 100 based on the rate pulse.

The receiving unit 112 includes a delay circuit, a switch such as a cross point switch, an amplifier circuit, an analog-to-digital (A/D) converter, an adder and the like and creates reflected wave data by performing various processing on reflected wave signals that have been received at the transducer elements of the ultrasound probe 100. The amplifier circuit performs gain correction by amplifying the reflected wave signals. The A/D converter converts the gain-corrected reflected wave signals from the analog format to the digital format and the delaying circuit provides a delay time that is required for determining reception directivity. The adder creates reflected wave data by adding the digitally converted reflected wave signals from the A/D converter. Through the addition processing, in one example, the adder emphasizes a reflection component from a direction in accordance with the reception directivity of the reflected wave signal. In the above described manner, the transmitting unit 111 and the receiving unit 112 respectively control transmission directivity during ultrasound transmission and reception directivity during ultrasound reception.

In the above described first embodiment, the cross point switch is directly connected to each of the outputs from the delay circuits that individually delay each of the output signals from the transducer elements. That is, the cross point switch selectively combines an individually delayed output signal from any single transducer element with any other such transducer outputs so as to dynamically form a desired element-by-element flexible subarray in beamforming.

Furthermore, the above described first embodiment the ultrasound diagnostic apparatus according to the current invention forms an image based upon a user input specifying a dynamic subarray. A touch input device 130 allows a user to input at least an image parameter value for generating an image. In another embodiment, an image parameter setting unit 130A receives at least an image parameter value for generating an image. The image processing unit 115 includes a separate subarray configuring unit 115A and provides a module or a function for defining dynamic subarrays and generating a dynamic subarray forming signal. In another embodiment, a separate subarray configuring unit is connected to the image parameter setting unit for defining dynamic subarrays and generating a dynamic subarray forming signal. An array has a predetermined number of transducer elements, and each of the transducer elements outputs a signal. A plurality of time-delay circuits is directly connected to the array for individually delaying each of the signals from the transducer elements to output time-delayed signals. At least one switch such as a crosspoint switch is connected to the time-delay circuits and the subarray configuring unit, and the switch connects any combination of the time-delayed signals to define the dynamic subarrays based, upon the dynamic subarray forming signal and to output dynamic subarray signals. Subsequently, a plurality of adders is connected to the switch for summing the dynamic subarray signals of the dynamic subarrays to output an added subarray signal. Finally, an image forming unit 115B forms the image based upon the added subarray signal.

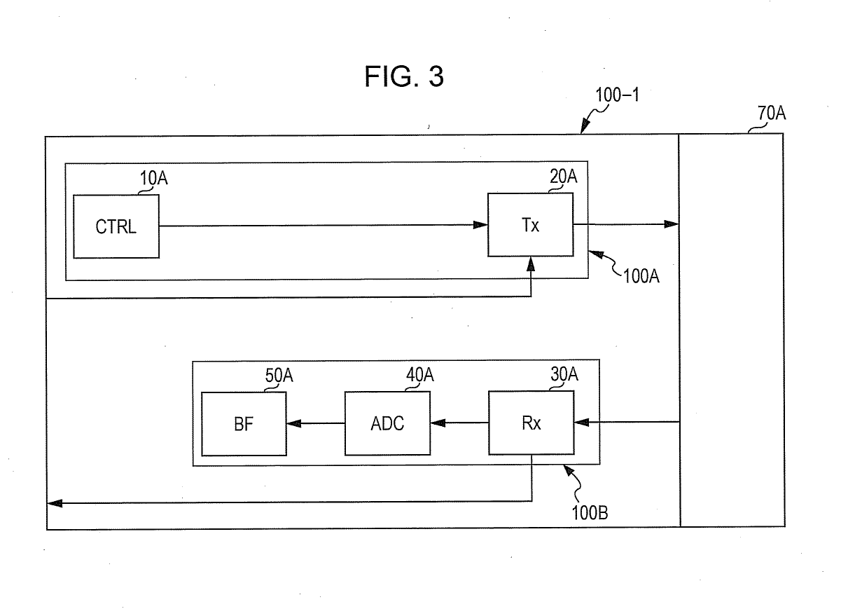

FIG. 3 is a diagram illustrating a second embodiment of the probe 100-1 according to the current invention. In general, the second embodiment of the probe 100-1 includes a transmission unit 100A, a receiving unit 100B and a transducer array unit 70A. The transmission unit 100A further includes a control unit (CTRL) 10A and a transmission circuit (Tx) 20A for controlling and producing ultrasound pulses from the transducer array unit 70A towards a region of interest in a patient or a subject. In this regard, the transmission circuit 20A receives control information from the control unit 10A and or an external source such as a processing unit as indicated by an incoming arrow.

The receiving unit 100B further includes a receiving circuit (Rx) 30A for receiving analog signals from the transducer array unit 70A, which receives the ultrasound echoes reflected from the region of interest in the patient. The receiving circuit 30A also optionally sends out the analog signals to an external source such as a processing unit as indicated by an outgoing arrow. The receiving unit 100B also further includes an analog-to-digital convertor (ADC) 40A for converting the analog electrical signals into digital signals which are then processed by a digital beam former unit (BF) 50A. The beam former unit 50A produces beam data, and this beam data is subsequently stored in a non-transitory local memory storage or medium 60A.

In the second embodiment, the transducer array unit 70A further includes a predetermined number of transducer elements that are dynamically configured in a certain size and array for the receiving circuit 30A. For example, the transducer elements are dynamically configured in a two-dimensional array, and a certain portion such as one or more rows of the transducer elements are dedicated to receive 1D imaging data while the rest of the transducer elements is dedicated to 3D/4D imaging volume data.

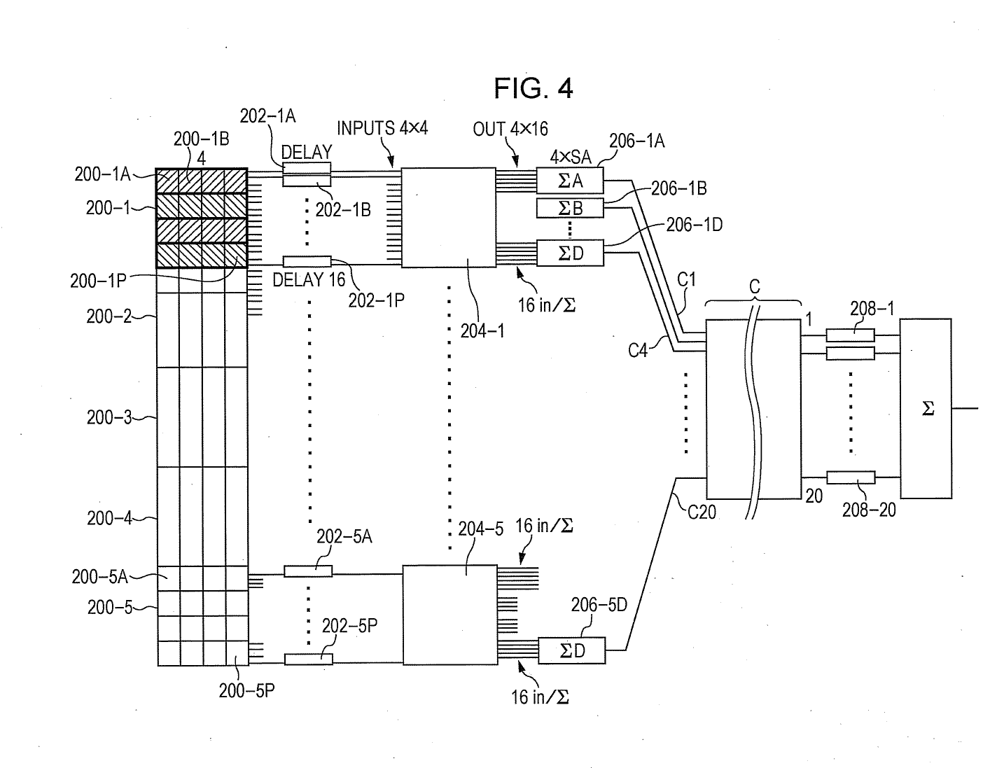

Now referring in particular to FIG. 4, a diagram illustrates additional components of the receiving unit 100B and the transducer unit 70A in the second embodiment of the probe 100-1 according to the current invention. In one implementation, the transducer unit 70A includes a transducer array 200 having a predetermined number of transducer elements 200-1A through 200-5P while the receiving unit 100B includes a corresponding number of time-delay circuits 202, a predetermined number of cross-point switches 204 and a predetermined number of adders 206. The eighty transducer elements 200-1A through 200-5P of the transducer array 200 are organized in a predetermined dimension of four rows by four columns of elements in the exemplary implementation. The transducer elements 200-1A through 200-5P receive the ultrasound echoes reflected from the region of interest in the patient for outputting analog signals. For each of the analog signals, a corresponding one of the time-delay circuits 202-1A through 202-5P is directly connected to delay the analog signal from one of the transducer elements 200-1A through 200-5P. The time-delay circuits 202-1A through 202-5P each process an appropriate amount of time delay on the analog signals to generate time-delayed signals. The appropriate delay is determined based upon a predetermined criterion such as directivity. Switches such as cross-point switches 204-1 through 204-5 are connected to the time-delay circuits 202-1A through 202-5P for achieving any combination of the time-delayed signals to define dynamic subarrays and to output dynamic subarray signals. Furthermore, a plurality of the adders 206-1A through 206-5D is connected to the switches 204-1 through 204-5 for summing the dynamic subarray signals of the dynamic subarrays to output an added subarray signal.

Still referring to FIG. 4, the transducer elements 200-1A through 200-5P in the two dimensional transducer array 200 are ultimately organized into dynamic subarrays on an element-by-element basis according to one embodiment of the current invention. For example, the two dimensional transducer array 200 has eighty elements, which are grouped into five exemplary subarrays (SAs) 200-1 through 200-5 as illustrated in FIG. 4. Each of the five exemplary SAs is organized by 4 by 4 transducer elements. That is, each of the SAs 200-1 through 200-5 has four transducer elements in both the elevation direction and the Azimuth direction. In the first SA 200-1, the sixteen transducer elements are individually referenced as 200-1A through 200-1P. By the same token, the sixteen transducer elements in the second SA 200-2 are individually referenced as 200-2A through 200-2P while the sixteen transducer elements in the third SA 200-3 are individually referenced as 200-3A through 200-3P. In the above exemplary implementation, although there are the five SAs 200-1 through 200-5, the number of SAs is not limited to a particular number according to the current invention. Similarly, in the exemplar implementation, although there are sixteen transducer elements in each of the five SAs 200-1 through 200-5, the SA size is not limited to a particular size according to the current invention. Furthermore, the SA size is optionally different among the SAs according to the current invention. Lastly, the SA configuration is also not limited to a particular shape even if each of the SAs includes the same number of the transducer elements.

In any embodiment according to the current invention, each of the transducer elements 200-1A through 200-5P in the array 200 is directly connected to a corresponding one of the time-delay circuits 202-1A through 202-5P. For example, the transducer elements 200-1A through 200-1P in the first SA 200-1 are respectively connected to the time-delay circuits 202-1A through 202-1P. Each of the transducer elements 200-1A through 200-5P in the array 200 generates an analog signal, and a corresponding one of the time-delay circuits 202-1A through 202-5P individually delays the analog signal by an appropriate amount of time before any other processing is performed on the analog signal. That is, a number of the time-delay circuits 202-1A through 202-5P is equal to the number of the transducer elements 200-1A through 200-5P for individually time-delaying the analog signals in the embodiment according to the current invention.

FIG. 4 further illustrates the element-by-element control in switching or connecting the time-delayed analog signals from the individual transducer elements in order to dynamically form SAs in one embodiment according to the current invention. In one embodiment, a separate switch is dedicated to each of the SAs for implementing the element-by-element control. In the above exemplary implementation, the embodiment has formed the five dynamic SAs 200-1 through 200-5 based upon the individually performed time-delay analog signals from the transducer elements 200-1A through 200-5P. In the same exemplary embodiment, each of the five cross-point switches 204-1 through 204-5 receives a set of sixteen time-delayed signals from a corresponding one of the SAs 200-1 through 200-5. Subsequently, each of the five cross-point switches 204-1 through 204-5 selectively combines the sixteen time-delayed signals from a corresponding SA on an element-by-element basis according to a predetermined rule or condition. The combination is not limited to a particular number of the sixteen time-delayed signals or a particular pattern since each of the five cross-point switches 204-1 through 204-5 optionally exerts control on an element-by-element basis and outputs combinations from the four sets of the sixteen signals. Consequently, each of the five cross-point switches 204-1 through 204-5 outputs four sets of the arbitrarily combined signals from the individual transducer elements within the corresponding SAs. Thus, each of the cross-point switches 204-1 through 204-5 forms the dynamic SAs based upon the arbitrarily combined sixty-four signals.

FIG. 4 also illustrates an additional control in switching or connecting the signals from individual transducer elements for dynamically forming SAs in one embodiment according to the current invention. In one embodiment, the adders 206-1A through 206-5D further add or sum the output sets of the combined signals based upon a predetermined rule according to the current invention. For example, the adders 206-1A through 206-1D each receive a corresponding set of the sixteen output signals from the cross-point switch 204-1. Each of the adders 206-1A through 206-1D outputs a single output channel signal in this exemplary implementation. Assuming that the system has twenty channels C1 through C20 as illustrated in a cable C, the adders 206-1A through 206-1D respectively output the four signals in the channels C1 through C4. By the same token, other sixteen adders 206-2A through 206-2D, 206-3A through 206-3D, 206-4A through 206-4D and 206-5A through 206-5D respectively output the sixteen signals in the channels C5 through C20. Consequently, the twenty adders 206-1A through 206-5D reduce the number of signals to meet the channel requirement of the system while the element-by-element control has been exerted through the above described process in forming dynamic SAs according to the current invention.

For the sake of simplicity, the illustrated system and cable requirements range from 10 to 20 channels as opposed to 32 to 256 channels in a typical system. Due to the independent control on the output signals from the transducer elements, the channel requirements are flexibly met based upon the dynamic SA formation in other embodiments according to the current invention. For example, for a system requiring ten channels in the cable, two adders such as 206-1A and 206-1B are used for each of the five cross-point switches 204-1 through 204-5 in the above described embodiment according to the current invention. In another implementation, the four adders are used while the two of the four adders output zero in order to meet the ten-channel requirement in the above described embodiment according to the current invention. Thus, a single probe having the above described dynamic SA-forming capability is used for different systems having various channel requirements.

The above embodiments merely illustrate exemplary implementations and are not limited to a particular number of the cross-point switches and or the adders to practice the current invention. For example, another embodiment is optionally implemented using a single cross-point switch which receives a number of the inputs that is the same as the number of the transducer elements in the array. By the same token, the above embodiment merely illustrates one exemplary implementation and is not limited to a particular number of output sets from the cross-point switches to practice the current invention.

In the above described second embodiments, the cross point switches are directly connected to each of the outputs from the delay circuits that individually delay each of the output signals from the transducer elements. That is, the cross point switch selectively combines an individually delayed output signal from any single transducer element with any other such transducer outputs within a dynamically formed subarray for beamforming. In other words, a subarray is formed in beamforming on an element-by-element basis in a flexible manner.

Furthermore, the diagram illustrates additional components of the receiving unit 100B and the transducer unit 70A with respect to the second embodiment of the probe 100-1 according to the current invention. The above described construction is not limited to the second embodiment and is optionally applicable to the first embodiment and other embodiments according to the current invention. The diagram is illustrated for the sake of simplicity and includes a significantly reduced number of elements of the 2D arrays to simplify the description of the embodiment.

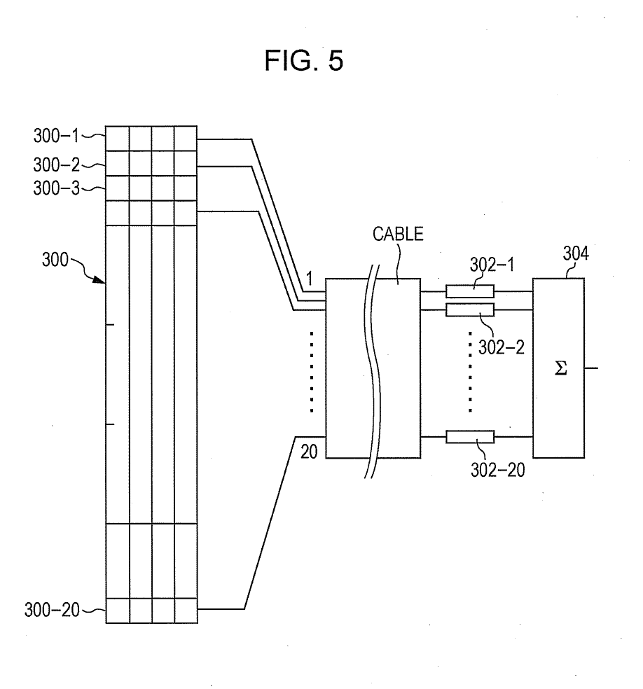

Now referring to FIG. 5, a diagram illustrates an exemplary one-dimensional equivalent embodiment with additional components of the receiving unit 100B and the transducer unit 70A in the second embodiment of the probe 100-1 according to the current invention. In general, one implementation of the receiving unit 100B includes a transducer array 300 having a predetermined number of SAs 300-1 through 300-20, a corresponding number of delay circuits 302-1 through 302-20 and an adder 304. The transducer elements 300-1 through 300-64 of the transducer array 300 are organized into twenty SAs 300-1 through 300-20, having a predetermined dimension of twenty rows with each row having four elements for receiving the ultrasound echoes reflected from the region of interest in the patient for outputting analog signals. The twenty SAs 300-1 through 300-20 are dynamically formed to implement a one-dimensional array equivalent in the probe 100-1. One dimensional probe shown in the diagram in FIG. 5 does not need the additional components such as the delay circuits, the cross point switch and the adders before the twenty outputs are matched with the 20-channel system requirement of the cable.

Still referring to FIG. 5, a corresponding one of the delay circuits 302-1 through 302-20 may be directly connected to each of the SAs 300-1 through 300-20 1 in an alternative embodiment. The time-delay circuits 302-1 through 302-20 respectively process an appropriate amount of time delay on the analog signals to generate time-delayed signals. The adder 304 is connected for summing the signals. The images from the above one-dimensional array are generated by dynamic beamforming. Thus, the first embodiment is capable of outputting data not only for generating a two-dimensional image from the two-dimensional array but also for generating a two-dimensional image from the simulated one-dimensional array. In other words, the illustrative embodiment has two-dimensional array transducer elements that can be electronically configured into one row, thus behaving like a 1D array.

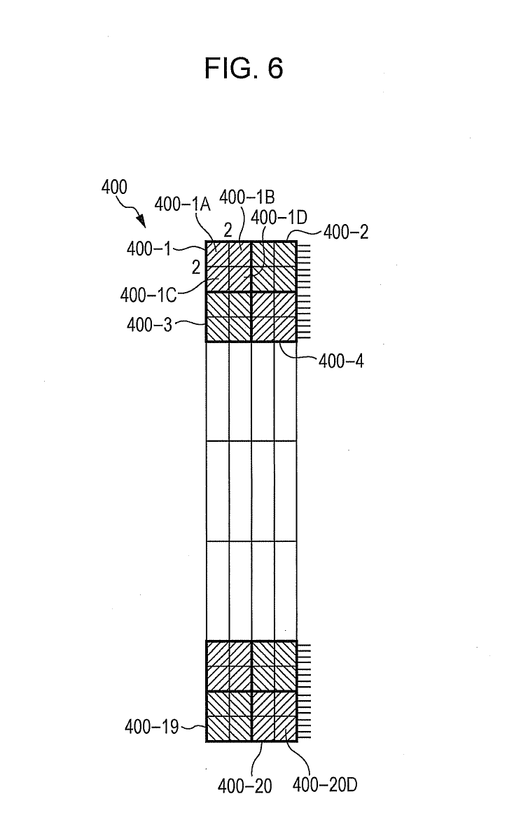

By the same token, three-dimensional imaging is implemented by dynamically forming the SAs using the above described embodiments according to the current invention. Now referring to FIG. 6, each of exemplary SAs is illustrated to have 2 by 2 transducer elements in the third embodiment according to the current invention. The eighty transducer elements 400-1A through 400-20D of the transducer array 400 are organized into twenty SAs 300-1 through 300-20, having a predetermined dimension of two rows and two columns. That is, each of the SAs 300-1 through 300-20 has four elements or two-by-two elements for receiving the ultrasound echoes reflected from the region of interest in the patient for outputting analog signals. For example, the SA 400-1 has the four elements 400-1A, 400-1B, 400-1C and 400-1D. By the same token, the four transducer elements in the second SA 400-2 are individually referenced as 400-2A through 400-2D while the four transducer elements in the third SA 400-3 are individually referenced as 400-3A through 400-3D. That is, each of the SAs 400-1 through 400-20 has two transducer elements in both the elevation direction and the Azimuth direction.

The signals from twenty SAs 400-1 through 400-20 are dynamically summed to implement a two-dimensional array equivalent to the probe 100-1. For the sake of simplicity, the diagram in FIG. 6 lacks the illustration of the additional components such as the delay circuits, the cross point switch and the adders before the eighty channel outputs are matched with the 20-channel system requirement of the cable. Although it is not illustrated, the element-by-element control is accomplished by switching or connecting the time-delayed analog signals from the individual transducer elements in order to dynamically form the two-dimensional SAs in the exemplary embodiment according to the current invention. In one embodiment, a separate switch is dedicated to each of the SAs for implementing the element-by-element control. In the above exemplary implementation, the embodiment has formed the twenty dynamic SAs 400-1 through 400-20 based upon the individually performed time-delay analog signals from the transducer elements 400-1A through 400-20D. In the same exemplary embodiment, a predetermined number of the cross-point switches receives a set of time-delayed signals from a corresponding one of the SAs 400-1 through 400-20. Subsequently, each of the cross-point switches selectively combines the time-delayed signals from a corresponding SA on an element-by-element basis according to a predetermined rule or condition. The combination is not limited to a particular number of the time-delayed signals or a particular pattern since each of the cross-point switches optionally exerts control on an element-by-element basis and outputs combinations from the twenty sets of the four signals. Consequently, each of the cross-point switches outputs a predetermined number of sets of the arbitrarily combined signals from the individual transducer elements within the corresponding SAs. Thus, each of the cross-point switches faints the dynamic SAs based upon the arbitrarily combined signals. Thus, the third embodiment is capable of outputting data to generate a three-dimensional image from the two-dimensional array according to the current invention.

Furthermore, with respect to the exemplary embodiment as illustrated in FIG. 6, a predetermined number of adders add or sum the output sets of the combined signals according to a predetermined rule according to the current invention. For example, the adders each receive a corresponding set of the output signals from one of the cross-point switches. Each of the adders outputs a single output channel signal in this exemplary implementation. In summary, the twenty adders optionally reduce the number of signals to meet the channel requirement of the system while the element-by-element control has been exerted through the above described process in forming dynamic SAs according to the current invention.

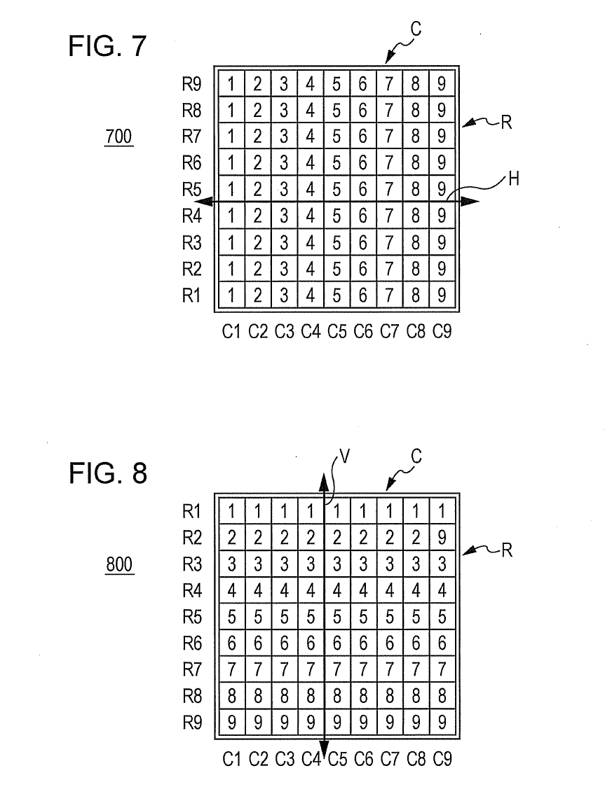

Now referring to FIG. 7, a diagram illustrates one exemplary subarray (SA) organization to improve image quality and applicability of two-dimensional (2D) arrays in one embodiment according to the current invention. In general, the transducer elements are grouped into various groups in a flexible manner, and the flexibly organized groups of the transducer elements are delayed and summed to produce multiple outputs. Ultimately, the transducer elements are controlled on an element-by-element basis for the above flexibly organized groups. For the sake of description, the exemplary embodiment has an array 700 with eighty-one transducer elements in nine rows R1 through R9 and nine columns C1 through C9. In the exemplary array 700, the nine-by-nine transducer elements are flexibly combined to form SAs along a predetermined horizontal direction as indicated by an arrow H for substantially improving 2D image quality.

Still referring to FIG. 7, the flexibly formed subarrays (SAs) are optionally formed by adding columns of the elements in the probe for generating data for horizontal or zero degree two-dimensional (2D) slices in one embodiment according to the current invention. Assuming that the scanning direction is substantially the same as the horizontal direction as indicated by the arrow H, each of the SAs consists of nine transducer elements in each one of the rows R1 through R9. That is, nine elements in each of the columns C1 through C9 are summed together for form a single SA. For example, the nine elements is are summed together in the column C1 while the nine elements 2s are summed together in the column C2. By the same token, a set of the vertically placed elements 3s through 9s is respectively summed in each of the columns C3 through C9. The SAs are flexibly formed by a certain device such as a cross point switch by combining the analog signals that have been individually delayed by a dedicated delaying circuit.

Now referring to FIG. 8, a diagram illustrates a second exemplary subarray (SA) organization to improve image quality and applicability of two-dimensional (2D) arrays in a second embodiment according to the current invention. In general, the transducer elements are grouped into various groups in a flexible manner, and the flexibly organized groups of the transducer elements are delayed and summed to produce multiple outputs. Ultimately, the transducer elements are controlled on an element-by-element basis for the above flexibly organized groups. For the sake of description, the exemplary embodiment has an array 800 with eighty-one transducer elements in nine rows R1 through R9 and nine columns C1 through C9. In the exemplary array 800, the nine-by-nine transducer elements are flexibly combined to form SAs along a predetermined vertical direction as indicated by an arrow V for substantially improving 2D image quality.

Still referring to FIG. 8, the flexibly formed subarrays (SAs) are optionally formed by adding rows of the elements in the probe for generating data for vertical or ninety-degree 2D slices in the second embodiment according to the current invention. Assuming that the scanning direction is substantially the same as the horizontal direction that is perpendicular to the arrow V, each of the SAs consists of nine transducer elements in each one of the rows R1 through R9. That is, nine elements in each of the rows R1 through R9 are summed together for form a single SA. For example, the nine elements 1s are summed together in the row R1 while the nine elements 2s are summed together in the row R2. By the same token, a set of the horizontally placed elements 3s through 9s is respectively summed in each of the rows R3 through R9. The SAs are flexibly formed by a certain device such as a cross point switch by combining the analog signals that have been individually delayed by a dedicated delaying circuit.

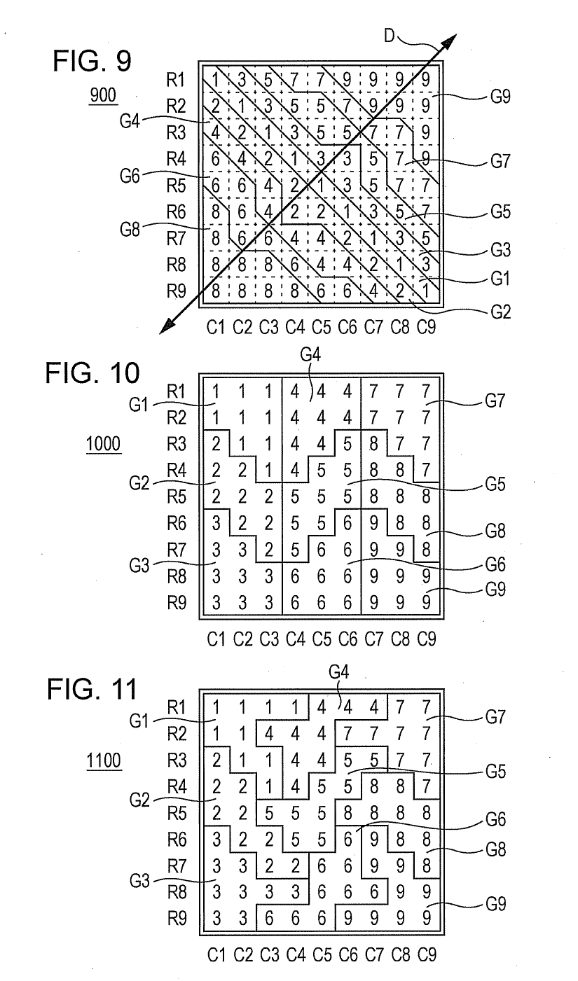

Now referring to FIG. 9, a diagram illustrates a third exemplary subarray (SA) organization to improve image quality and applicability of two-dimensional (2D) arrays in a third embodiment according to the current invention. In general, the transducer elements are grouped into various groups in a flexible manner, and the flexibly organized groups of the transducer elements are delayed and summed to produce multiple outputs. Ultimately, the transducer elements are controlled on an element-by-element basis for the above flexibly organized groups. For the sake of description, the exemplary embodiment has an array 900 with eighty-one transducer elements in nine rows R1 through R9 and nine columns C1 through C9. In the exemplary array 900, the nine-by-nine transducer elements are flexibly combined to form nine SAs along a predetermined diagonal direction as indicated by an arrow D for improving 2D image quality.

Still referring to FIG. 9, the flexibly formed subarrays (SAs) are optionally used for generating data for diagonal or forty-five-degree 2D slices in the third embodiment according to the current invention. Assuming that the scanning direction is substantially the same as the horizontal direction that is forty-five degree to the arrow D, each of the SAs consists of a certain number of transducer elements in each one of the nine SAs G1 through G9. For example, the first through ninth SAs G1 and G9 each is formed by combining nine transducer elements. In the first SA G1, the nine transducer elements are all marked as 1. By the same token, the nine transducer elements are marked by the corresponding number of the second through ninth SAs G2 through G9. Although the number of the transducer elements is the same among the nine SAs G1 through G9, the shape of the nine SAs G1 through G9 varies from one SA to another SA. The SAs G1 through G9 are flexibly formed by a certain device such as a cross point switch by combining the analog signals that have been individually delayed by a dedicated delaying circuit.

Now referring to FIG. 10, a diagram illustrates a fourth exemplary subarray (SA) organization to improve image quality and applicability of two-dimensional (2D) arrays in a fourth embodiment according to the current invention. In general, the transducer elements are grouped into various groups in a flexible manner, and the flexibly organized groups of the transducer elements are delayed and summed to produce multiple outputs. Ultimately, the transducer elements are controlled on an element-by-element basis for the above flexibly organized groups. For the sake of description, the exemplary embodiment has an array 1000 with eighty-one transducer elements in nine rows R1 through R9 and nine columns C1 through C9. In the exemplary array 1000, the nine-by-nine transducer elements are flexibly combined to form nine SAs in imaging data having random edges in one direction in order to lower side lobes improve 2D image quality.

Still referring to FIG. 10, the flexibly formed subarrays (SAs) are optionally used for generating data in 2D slices in another embodiment according to the current invention. Assuming that the data has random edges in a predetermined direction, each of the SAs consists of a certain number of transducer elements in each one of the nine SAs G1 through G9. For example, the first through ninth SAs G1 and G9 each is formed by combining nine transducer elements. In the first SA G1, the nine transducer elements are all marked as 1. By the same token, the nine transducer elements are marked by the corresponding number of the second through ninth SAs G2 through G9. Although the number of the transducer elements is the same among the nine SAs G1 through G9, the shape of the nine SAs G1 through G9 varies from one SA to another SA. The SAs G1 through G9 are flexibly formed by a certain device such as a cross point switch by combining the analog signals that have been individually delayed by a dedicated delaying circuit.

Now referring to FIG. 11, a diagram illustrates a fifth exemplary subarray (SA) organization to improve image quality and applicability of two-dimensional (2D) arrays in a fifth embodiment according to the current invention. In general, the transducer elements are grouped into various groups in a flexible manner, and the flexibly organized groups of the transducer elements are delayed and summed to produce multiple outputs. Ultimately, the transducer elements are controlled on an element-by-element basis for the above flexibly organized groups. For the sake of description, the exemplary embodiment has an array 1100 with eighty-one transducer elements in nine rows R1 through R9 and nine columns C1 through C9. In the exemplary array 1100, the nine-by-nine transducer elements are flexibly combined to form nine SAs in imaging data having random edges in both directions in order to improve 2D image quality.

Still referring to FIG. 11, the flexibly formed subarrays (SAs) are optionally used for generating data in 2D slices in another embodiment according to the current invention. Assuming that the data has random edges in predetermined two directions, each of the SAs consists of a certain number of transducer elements in each one of the nine SAs G1 through G9. For example, the first through ninth SAs G1 and G9 each is formed by combining nine transducer elements. In the first SA G1, the nine transducer elements are all marked as 1. By the same token, the nine transducer elements are marked by the corresponding number of the second through ninth SAs G2 through G9. Although the number of the transducer elements is the same among the nine SAs G1 through G9, the shape of the nine SAs G1 through G9 varies from one SA to another SA. The SAs G1 through G9 are flexibly formed by a certain device such as a cross point switch by combining the analog signals that have been individually delayed by a dedicated delaying circuit.

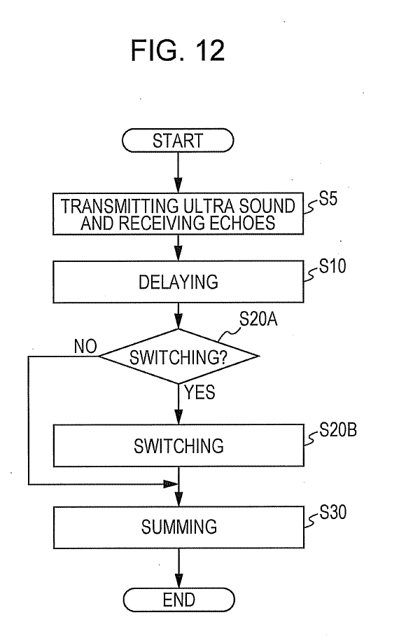

FIG. 12 is a flow chart illustrating the steps or acts involved in one embodied process with respect to the probe according to the current invention. The embodied process in the probe starts when ultrasound pulses are transmitted towards a region of interest and the reflected ultrasound echoes are received from the region of interest in a step S5. In one embodied process, the above described transmission and reception are repeated while the received image data is concurrently being displayed. The reflected ultrasound echoes are received at transducer elements in an array. The transducer elements generate analog signals based upon the received ultrasound echoes. The transducer elements are provided in a two dimensional transducer array. Each of the transducer elements in the array is directly connected to a corresponding one of the time-delay circuits. In one exemplary process according to the current invention, the analog signals are individually delayed in a step S10 by an appropriate amount of time before any other steps is performed on the analog signal. In one implementation, a number of the time-delay circuits is equal to the number of the transducer elements for individually time-delaying the analog signals in the embodiment according to the current invention.

Still referring to FIG. 12, the time delayed signals are now dynamically grouped into flexible subarrays in a step S20B in one exemplary process according to the current invention. That is, if it is determined in a step S20A that the switching step S20B takes place, a two dimensional transducer array is optionally organized into dynamic subarrays on an element-by-element basis in the step S20B according to one embodiment of the current invention. For example, the two dimensional transducer array has eighty elements, which are grouped into five exemplary subarrays (SAs), and each of the five exemplary SAs is organized by 4 by 4 transducer elements. That is, each of the five SAs has four transducer elements in both the elevation direction and the Azimuth direction. In the steps S20A and S20B, the element-by-element control is exerted in switching or connecting the time-delayed analog signals from the individual delayed circuits in order to dynamically form SAs in one exemplary process according to the current invention.

FIG. 12 also illustrates an additional control step in switching or connecting the signals from individual transducer elements for dynamically forming SAs in one exemplary process according to the current invention. In one exemplary process, the output sets of the combined signals from the step S20B are further added or summed based upon a predetermined rule in a step S30 according to the current invention. For example, the adders each receive a corresponding set of the output signals from a cross-point switch. Each of the adders outputs a single output channel signal in this exemplary implementation: In one implementation of the step S30, the adders reduce the number of signals to meet the channel requirement of the system while the element-by-element control has been exerted through the above described process in forming dynamic SAs according to the current invention.

The above process merely describes an exemplary process and is not limited to a particular implementation such as in a number of the cross-point switches and or the adders to practice the current invention. By the same token, the above steps merely illustrate one exemplary implementation and are not limited to a particular number of output sets from the cross-point switches to practice the current invention.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope of the inventions.

What is claimed is:

1. An ultrasound probe, comprising:
an array of a predetermined dimension having a predetermined number of transducer elements, each of the transducer elements outputting a signal;
a plurality of time-delay circuits directly connected to said array for individually delaying each of the signals from the transducer elements to output time-delayed signals;
at least one switch connected to said time-delay circuits for connecting any combination of the time-delayed signals to define dynamic subarrays and to output dynamic subarray signals; and
a plurality of adders connected to said switch for summing the dynamic subarray signals of the dynamic subarrays to output an added subarray signal.

2. The ultrasound probe according to claim 1 wherein each of said time-delay circuits delays by down-converting the signal to a base band and or up-converting the base band.

3. The ultrasound probe according to claim 1 wherein said switch is a cross-point switch configured to form the dynamic subarrays that are not all equal in configuration.

4. The ultrasound probe according to claim 1 wherein said switch is a cross-point switch configured to form the dynamic subarrays that are not all equal in number of the elements.

5. The ultrasound probe according to claim 1 wherein the dynamic subarrays have a combination of a variable elevation edge and a variable lateral edge.

6. The ultrasound probe according to claim 1 wherein said adders are cable of outputting a reduced number of the dynamic subarray signals.

7. The ultrasound probe according to claim 6 wherein the reduced number of the dynamic subarray signals is matched with a predetermined number of system channels connecting a probe.

8. The ultrasound probe according to claim 1 further comprising a probe housing for housing said array, said time-delay circuits, said switch and said adders.

9. The ultrasound probe according to claim 1 according to claim 1 wherein said switch is a cross-point switch that connects any one of the elements to any distantly located one of the elements in said array.

10. A method of dynamically defining a subarray in the ultrasound array system,
comprising:
providing in a probe an array a predetermined number of elements in a predetermined dimension;
outputting a signal from each of the elements in the array;
delaying the signal directly from each of the elements in the array to output time-delayed signals;
connecting any combination of the time-delayed signals in the probe for selectively defining dynamic subarrays and outputting dynamic subarray signals; and
summing the dynamic subarray signals of the dynamic subarrays in the probe to output an added subarray signal.

11. The method of dynamically defining a subarray in the ultrasound array system according to claim 10 wherein the signal is delayed by down-converting the signal to a base band or up-converting the base band.

12. The method of dynamically defining a subarray in the ultrasound array system according to claim 10 wherein the dynamic subarrays are not all equal in configuration.

13. The method of dynamically defining a subarray in the ultrasound array system according to claim 10 wherein the dynamic subarrays are not all equal in number of the elements.

14. The method of dynamically defining a subarray in the ultrasound array system according to claim 10 wherein the dynamic subarrays have a combination of a variable elevation edge and a variable lateral edge.

15. The method of dynamically defining a subarray in the ultrasound array system according to claim 10 wherein a reduced number of the dynamic subarray signals is outputted after said summing.

16. The method of dynamically defining a subarray in the ultrasound array system according to claim 15 wherein the reduced number of the dynamic subarray signals is matched with a predetermined number of system channels connecting a probe.

17. The method of dynamically defining a subarray in the ultrasound array system according to claim 10 wherein any one of the elements is connected to any distantly located one of the elements in the array in said connecting.

18. An ultrasound diagnostic apparatus, comprising:
an image parameter setting unit for inputting at least an image parameter value for generating an image;
a subarray configuring unit connected to said image parameter setting unit for defining dynamic subarrays and generating a dynamic subarray aiming signal;
an array having a predetermined number of transducer elements, each of the transducer elements outputting a signal;
a plurality of time-delay circuits directly connected to said array for individually delaying each of the signals from the transducer elements to output time-delayed signals;
at least one switch connected to said time-delay circuits and said subarray configuring unit for connecting any combination of the time-delayed signals to define the dynamic subarrays based upon the dynamic subarray forming signal and to output dynamic subarray signals;
a plurality of adders connected to said switch for summing the dynamic subarray signals of the dynamic subarrays to output an added subarray signal; and
an image forming unit connected to said adders for forming the image based upon the added subarray signal.
